(19) United States
(12) Patent Application Publication
(10) Pub. No.: US 2005/0043639 A1
(43) Pub. Date: Feb. 24, 2005

(54) Method for decreasing mortality of hemodialysis patients

(76) Inventor: Robert E. Fischell, Dayton, MD (US)

Correspondence Address: Dr. Robert E. Fischell, 14600 Viburnum Drive, Dayton, MD, 21036, US

(21) Appl. No.: 10/645,147

(22) Filed: Aug. 22, 2003

(51) Int. Cl. A61B 5/0402

(52) U.S. Cl. 600/509; 600/515

(57) ABSTRACT

Disclosed is a means and method to decrease mortality and morbidity of patients who regularly undergo either hemodialysis (typically) at a hospital or who undergo peritoneal dialysis at home. The present invention utilizes an implantable system that can detect early signs of a heart attack by noting changes in the electrogram signal from an electrode placed near the apex of the right ventricle of the patient's heart. What is specifically noted from the electrogram for the early detection of a heart attack is a shift in the ST segment compared to a baseline level of the ST segment that was placed in the implanted device's memory at an earlier time. The implanted system is also in contact with an externally located alarm means that also can alert the patient if a heart attack occurs.

### FIELD OF USE

This invention is in the field of methods and devices for warning dialysis patients that they are having a heart attack so that they can take actions to save their life.

### BACKGROUND OF THE INVENTION

Patients who undergo hemodialysis have an extraordinarily high mortality rate from myocardial infarction; i.e., a heart attack. One reason for this high death rate for dialysis patients is that approximately 45% of these patients have diabetes and most of those patients have silent ischemia; i.e., when an acute myocardial infarction (AMI) occurs, they have no symptom that they can detect. Silent ischemia significantly increases the mortality rate for such patients.

The medical literature has indicated that the annual mortality rate from a second heart attack for those dialysis patients who have had a first heart attack is 41%. The rate is even higher for dialysis patients who are elderly or who have diabetes. At the present time there is no means or method to provide an early warning of a heart attack for such dialysis patients. This is especially true for those patients who have had an AMI (which is a heart attack) without any detectable symptom; typically because of silent ischemia.

### SUMMARY OF THE INVENTION

The present invention utilizes an implantable system that can detect early signs of a heart attack by noting the electrogram signal from an electrode placed near the apex of the right ventricle of the patient's heart. Unlike an electrocardiogram which is sensed by electrodes placed on a patient's skin, the electrogram is the electrical signal from inside the body and particularly from inside the heart. What is specifically noted from an electrogram for the early detection of a heart attack is a shift in the ST segment compared to a baseline level of the ST segment that was placed in the implanted device's memory at an earlier time. The means and methods for using such a system are described in U.S. Pat. Nos. 6,112,116, 6,272,379, 6,468,263 and 6,609,023, all of the preceding patents being included herein by reference.

The most important object of the present invention is teach a novel method to decrease mortality from a heart attack for those patients who are being treated by dialysis.

This and other objects and advantages of this invention will become obvious to a person of ordinary skill in this art upon reading the detailed description of this invention including the associated drawing as presented herein.

### BRIEF DESCRIPTION OF THE DRAWING

FIG. 1. is a sketch of a dialysis patient who has an implanted system for the earliest possible detection of a heart attack.

### DETAILED DESCRIPTION OF THE INVENTION

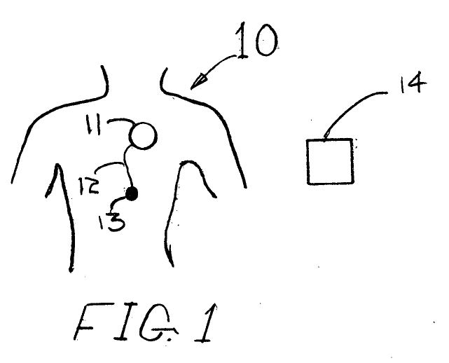

FIG. 1 illustrates a dialysis patient who has within his or her body an implanted system 10 for prompt detection of an AMI. The implanted system consists of an electrogram monitor 11 that receives an electrogram signal through an electrical lead 12 that is connected to an electrode 13 that is typically placed within the patient's right ventricle. It should be understood that the electrode 13 could also be placed under the skin, typically in the region of the body that is near to the heart. A metal can of the electrogram monitor 11 would act as the second electrode for the detection of an electrogram signal. The system 10 is described in detail in one or more of the references that are included herein by reference. Also described in the cited references is a description of various external alarm means 14 that work in conjunction with an alarm means within the monitor 11 to provide an early warning for the dialysis patient that he or she is having a heart attack. An external alarm means 14 is shown in FIG. 1 as a separate box located externally to the patient. Such an external alarm means 14 and the use of remote sites for assisting the heart attack victim are described in the U.S. patents as cited above. Furthermore, the use of an implanted pacemaker or implanted defibrillator that includes the ability to detect an AMI is also a part of the concept of this invention.

An important purpose of the external alarm means 14 is to receive an alarm signal from the monitor 11 and trigger an audio means within the external alarm means 14 to wake the patient up if that patient is sleeping. The audio communication from the external alarm means 14 could also include a pre-recorded advice message for the patient. Another audio signal to come from the external alarm means 14 could originate from a remotely located diagnostic center where a medical practitioner who has been made aware of the patient's heart attack through the system of FIG. 1, has two-way voice contact with the patient.

An algorithm that can be used for determining if a heart attack is occurring is described in detail in the cited reference that is U.S. Pat. No. 6,609,023.

The method for using the present invention is as follows:
a) determining if the patient has been connected to a system that performs dialysis;
b) implanting into that patient a system for early warning of a heart attack by means of noting an ST segment shift in that patient's electrogram;
c) sounding an alarm from within the implanted system to warn the patient that he or she is having a heart attack.

An additional step would be:
d) having an external alarm system in close proximity to the dialysis patient so that it would receive a signal from the implanted system indicating that a heart attack has been detected.

Still another step would be:
e) having the external system provide an audio alarm when a heart attack is detected by the implanted system, the audio alarm being detectable by the dialysis patient who is having a heart attack.

Still another step would be:
f) having the external alarm system communicate with a remotely located diagnostic center, the diagnostic center having a medical practitioner who would have voice contact with the patient after the implanted system has indicated that a heart attack has been detected.

It should be noted that the method and devices described herein could be used for patients who are treated at a hospital, clinic or medical center with hemodialysis or who treat themselves at home by means of peritoneal dialysis. It should also be noted that the method described herein is of particular importance for diabetic dialysis patients, particularly those who have had a first heart attack.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

1. A method for providing an alarm signal to alert a dialysis patient that he or she is having a heart attack, the method including the following steps:
a) determining if the patient is a dialysis patient who has been connected to a system that performs dialysis;
b) implanting into the patient a system for early warning of a heart attack by means of noting an ST segment shift in that patient's electrogram;
c) sounding an alarm from within the implanted system to warn the patient that he or she is having a heart attack.

2. The method of claim 1 including the additional step of having an external alarm system in close enough proximity to the dialysis patient so that it would receive a signal from the implanted system indicating that a heart attack has been detected.

3. The method of claim 2 including the additional step of having the external system provide an audio alarm when a heart attack is detected by the implanted system, the audio alarm being able to be detected by the dialysis patient who is having a heart attack.

4. The method of claim 2 including the additional step of having the external alarm system being capable of communicating with a remotely located diagnostic center, the diagnostic center having a medical practitioner who would have voice contact with the patient after the implanted system has indicated that a heart attack has been detected.

5. The method of claim 1 including the additional step of determining if the patient has diabetes.

6. The method of claim 1 including the additional step of determining if the patient has had a prior heart attack.

7. The method of claim 1 including the additional step of determining if the patient has diabetes and has had a prior heart attack.

8. The method of claim 1 including the additional step of determining if the patient has an implanted pacemaker of defibrillator.
